(19) United States
(12) Patent Application Publication
(10) Pub. No.: US 2014/0221740 A1
(43) Pub. Date: Aug. 7, 2014

(54) WIRELESS ENDOSCOPIC SURGICAL DEVICE

(71) Applicants: Paul John Kawula, Sunnyvale, CA (US); John William Doyle, Milwaukie, OR (US); Sachiko Hattori, Sunnyvale, CA (US)

(72) Inventors: Paul John Kawula, Sunnyvale, CA (US); John William Doyle, Milwaukie, OR (US); Sachiko Hattori, Sunnyvale, CA (US)

(21) Appl. No.: 13/759,920

(22) Filed: Feb. 5, 2013

(51) Int. Cl. A61B 1/00; A61B 1/06; A61B 1/04

(52) U.S. Cl. CPC A61B 1/00016; A61B 1/042; A61B 1/0684
USPC 600/109

(57) ABSTRACT

A wireless endoscopic surgical device used for minimally invasive procedures comprises a handheld component and a separate power module. The handheld component consisting of a handle and a conduit houses a wireless imaging system and a single LED light source. The imaging system comprises a wireless camera coupled to an optical assembly. Both the intensity of the LED and the camera action can be controlled by a battery-operated power module. The handle and conduit are designed to accommodate surgical tools.

### BACKGROUND

Endoscopes are used to view the inside of the body through a small incision during minimally invasive surgery. For this section, the rigid type used in endoscopic vessel harvesting (EVH) will be discussed, but similar features and therefore issues could exist in other surgeries using endoscopes as part of a system.

A rigid endoscope system comprises the following: the endoscope itself, that is, a long tubular metallic conduit which would contain optical fiber bundles that extend from the proximal end in a handle to the distal viewing tip. A light source cable connects to the proximal end to provide light for viewing, and the resultant image is carried through a separate optical system (lenses), back to an external camera at the proximal end. Images may be processed and stored in the camera or sent to a monitor for viewing, after being processed in an external video processing box.

For EVH, a single cannula is inserted into the leg opening (above the knee), through which both the endoscope as well as surgical tools are introduced. The procedure is used to harvest vessels (for bypass graft material) for coronary artery bypass graft (CABG) surgeries. Compared with open surgery, which can result in long, painful recovery and a foot-long scar, an endoscopic procedure is definitely preferred for patient comfort.

### Problem Statement

However, these systems can have issues. First is failure of a component of a system, especially if it is a re-processable item and the other is the bulk or unwieldy nature of a system.

When a component fails and it is a fairly expensive piece of capital equipment, the result can be problematic.

Endoscopes are delicate instruments, and can become damaged through handling associated with repeated use, cleaning, and re-sterilization. Owing to the cost, most cardiac Operating Rooms (OR)s would not have many back-up scopes.

Damage or failure in a scope discovered during system set up could trigger not only repair work but if no back-up scopes are immediately available, this could also result in conversion to an open procedure. In EVH, this becomes an FDA-reportable incident requiring a report that needs to be filed and followed up. The procedure now becomes a regular surgical procedure with associated patient discomfort and increase in cost of care.

To avoid such possible scenarios, endoscopes are cleaned, re-sterilized, and stored with great care. Scope use is tracked, and they are maintained and upgraded as necessary. Education and training in scope care as well as the actual cleaning processes take staff time.

Light source boxes for the scopes, although not as delicate, also need to be maintained as capital equipment. All these processes take additional time and resources adding to the cost of the procedure or as overhead to the hospital.

The set up may also have some inherent safety issues. The external light source box can attain high temperatures and cause burns if mishandled. In some surgery cases, the fiber optic light cable and camera power cord stretching from the equipment-laden tower to the patient table can cause clutter and become a potential safety hazard with many operators and technicians working in a small OR. This issue of having cables and cords between the patient table and the side of the room has been one of the reasons mentioned in literature promoting wireless connectivity within the OR.

Even if all the components are functional, assembling them takes time, and the result might not always be an optimal surgical system. An assembled endoscope system for EVH, for example, can be rather bulky.

If some or all of the functions of an endoscope system could be integrated and be available to the operator as one device, it could alleviate some of these issues. Similar situations probably exist for other endoscopic procedures as well.

Possible Solution

There have been designs that have resolved some of the issues of the endoscope systems mentioned above.

For example, in one version of an endoscope system, captured images are sent wirelessly from the video processor to the monitor (manufacturer's web-site). The camera and endoscope, however, appear to be still tethered to the video processor box on the tower (collection of power control, light source box on a cart in the OR). The fiber optic cable would also still be there. Therefore, although the image is no longer transmitted over a wire to the monitor, the overall bulkiness of the system is not resolved.

The use of wireless image transmittance has been increasingly common in the medical industry. A sample of patents that utilize wireless technology in endoscope systems can be seen as follows. Objectives of using wireless may be two-fold: to enhance remote access and to reduce bulk of system.

Wireless connections are indicated for endoscope use in remote locations (U.S. Pat. No. 7,048,686 B2) or for connecting several endoscopes to a central control unit (U.S. Pat. No. 6,902,529 B2).

One patent in particular mentions how a wireless endoscope might be useful in dental procedures for providing images of areas that are hard to access. (U.S. Pat. No. 7,030,904 B2). An area that has seen an effective mix of wireless technology and light source size reduction would be the in-vivo imaging or pill camera industry. Several companies describe how these work: (U.S. Pat. Nos. 7,996,067 B2; 7,998,059 B2; 8,165,374 B1). These miniaturized cameras with internal light sources are developed with similar goals (decrease in bulk of a system), but are made specifically for observation of the gastro-intestinal tract.

Internalizing a light source has been achieved by the use of light emitting diodes (LED)s. Often multiple LEDs are used to achieve the high intensity needed for endoscopes (U.S. Pat. No. 6,260,994 B1), but some utilize a single LED (U.S. Pat. No. 7,976,459 B2), and concern for efficient coupling of the light guide bundle to the LED has led one group to direct contact of the LED to the light guide without lenses or mirrors (U.S. Pat. No. 7,198,397 B2).

Batteries are used to drive LEDs in some endoscope systems (U.S. Pat. Nos. 6,260,994 B1; 8,152,715 B2); however, many LEDs seem to be connected to external power sources. Therefore, although both wireless and LED technologies can offer size reduction in devices, there does not seem to have been a combination of the two that has led to smaller endoscopes for general use.

Optics is the most critical part in an endoscope. However, aside from improving optical image quality, the essential elements of what is used for transferring light from the source to the target and the resultant image back to the camera have not changed much. Light and images are transferred by combinations of fiber optic bundles, lenses and mirrors.

Fiber optic bundles that are most commonly used can be cost effective, however, can display optical artifact issues derived from packing density that can worsen with length. It is likely for this reason that in many rigid endoscopes, gradient-index (GRIN) lenses have been used. However, GRIN lenses are long, rigid lenses, limited in the length they can be made, and can be costly. Perhaps this is one of the reasons that the current endoscopes for EVH (rigid) are made to be re-usable. If a more economic way to transfer images can be found, the scope could be made disposable, leading to lower costs.

The brief review of some of the technologies in an endoscope system reveals that there are many ways in which the system can be improved to offer smaller, lighter devices. However, to date, there has been no major change in rigid endoscope systems.

In addition to integrating the components of the endoscope system, for EVH, a surgical tool needs to be accommodated. Therefore, this would call for not only a smaller endoscope, but also a conduit portion that allows existent surgical tools to be introduced. To this end, the disclosed device has been conceived as a stand-alone device for the EVH procedure around which the optics and electronics have been designed.

It is expected that the disclosed system can make the EVH procedure simpler for the operator and by extension make the patient more comfortable. It is also expected that there would be sizable cost savings for the hospital as costly capital equipment (scope and light source) need not be maintained, and associated costs tied to reprocessing the scope (staff time, cleaning and sterilization costs) are eliminated.

In the current invention, a system is described that integrates the illuminating system and imaging system (camera) into a single conduit-handle part with a separate power and control part that drives the system.

The system would come in one disposable package, ready for use.

There would be no need to assemble components or attach to outside equipment. ORs could keep an inventory of these systems for procedures. Should any damage be discovered, another package could be opened without delay in procedure or conversion to open surgery.

As these systems are disposable, there would be no need to educate staff members in special cleaning, sterilization, or maintenance procedures, which saves time and resources for a hospital. Single use devices such as this also makes for simpler inventory control as there would be no need to coordinate capital equipment service agreements with vendors.

In this device/system, the illuminating system comprises a single High Brightness Light Emitting Diode (HB LED) contained inside a handle. Light transfers down the conduit through a light pipe to the target in the body. The formed image is transferred through fiber optic bundles in an in-line or folded optical cavity configuration in the conduit back to a camera that resides inside the handle. The image is then transmitted wirelessly to an outside monitor. There are thus, no fiber optic cables needed for a light source and no camera cords needed to send the image back to an image/video processor box.

The power and control part of the system (Power Control Module=PCM) is connected to the proximal end of the handle by a single cord. The PCM circuitry controls the power to the camera, and controls the power and intensity of the LED. The PCM box could be placed on the operating table, keeping the only cord close to the operator and not across the OR table or across the room.

The internalized camera, wireless transmission of the image, and optics designed around a device configuration enabled the overall size of the device to be small. Compared with an assemblage of cannula, camera, and associated cables and cords of a conventional system, a conduit with a handle is much more compact and therefore expected to be easier for the operator to manipulate during the procedure.

As minimally invasive procedures differ depending on the location of the anatomy where it is used, not all parts of the invention may be applicable to all endoscopic procedures. However, it is estimated that many parts of the invention disclosed here might be useful in other procedures as well.

Finally, as wireless connectivity becomes more prevalent in hospital environments both internally and externally, the use of wireless transmission of data is expected to become the norm. The invention disclosed describes an embodiment in which the data is an optical image. Other data (temperature, gas levels, fluorescence signals etc.) are expected to be also transmittable with the appropriate sensors in modified configurations.

### BRIEF SUMMARY

A disposable, integrated wireless endoscope system is disclosed that contains the following components in an integrated design:

A single High Brightness LED (HB LED) light source and light pipe are housed in the handle and the metal conduit that comes out of the handle. Light is transferred to the target tissue structure in the body at the distal end to illuminate a target and form an image. The image is transferred back through an imaging system that resides in the metal conduit and handle back to the proximal portion of the system, into a wireless camera.

The image is then transmitted wirelessly from the camera to a monitor or computer in the room. A power cord attaches the handle and conduit portion of the system to a power control box that is run on three C-cell batteries.

Another aspect of the endoscope system is the use of segmented coherent fibers (CF) in the imaging system. By designing an optical system to fit an integrated design, a relatively low cost, disposable endoscope system can be made.

### BRIEF DESCRIPTION OF THE DRAWINGS

FIG. 1a is an overall system layout of a first embodiment 1 of the invention, showing the major components and their interconnections.

FIG. 1b is an overall system layout of a second embodiment 81 of the invention, showing the major components and their interconnections.

FIG. 1c is an overall system layout of a third embodiment 101 of the invention, showing the major components and their interconnections.

FIG. 1d is an overall system layout of a fourth embodiment 181 of the invention, showing the major components and their interconnections.

FIG. 1e is an overall system layout of a fifth embodiment 401 of the invention, showing the major components and their interconnections.

FIG. 1f is an overall system layout of a sixth embodiment 411 of the invention, showing the major components and their interconnections.

FIG. 2 details a first embodiment of the endoscopic device 2 (see FIG. 1a).

FIG. 3 details a second embodiment of the endoscopic device 82 (see FIG. 1b).

FIG. 4 details a third embodiment of the endoscopic device 102 (see FIG. 1c).

FIG. 5 details a fourth embodiment of the endoscopic device 182 (see FIG. 1d).

FIG. 6 details a first embodiment of the imaging assembly 260.

FIG. 7 details a second embodiment of the imaging assembly 250.

FIG. 7a details a partial assembly of the first embodiment of the endoscopic device 2 (see 2 in FIG. 1a and FIG. 2).

FIG. 7b details a partial assembly of the second embodiment of the endoscopic device 82 (see 82 in FIG. 1b and FIG. 3).

FIG. 7c details a partial assembly of the third embodiment of the endoscopic device 102 (see 102 in FIG. 1c and FIG. 4).

FIG. 7d details a partial assembly of the fourth embodiment of the endoscopic device 182 (see 182 in FIG. 1d and FIG. 5).

FIG. 7e details a partial assembly of the fifth embodiment of the endoscopic device 402 (see 402 in FIG. 1e).

FIG. 7f details a partial assembly of the sixth embodiment of the endoscopic device 412 (see 412 in FIG. 1f).

FIG. 7g provides two views of a distal lumen baffle 150 and an optically transparent shield 151

FIG. 8 is a block diagram of the power and control systems (PCS) (315 and 316, respectively in FIG. 8) for the six embodiments of the invention (see 3 in FIG. 1a through FIG. 1f).

FIG. 9 is a sample of possible pulse-width modulated waveforms (A through E) that would represent the power applied to a high brightness light emitting diode (HB LED) in order to control the intensity of said HB LED.

FIG. 10 is a drawing of the inside of the enclosure 350 for the power and control module (PCM) 3 (in FIG. 1a through FIG. 1f) in the drawings of the six embodiments of the invention (in FIG. 1a through FIG. 1f).

FIG. 11 is a drawing of what the outside of the enclosure 350 would look like for the power and control module (PCM) 3 (in FIG. 1a through FIG. 1f) in the six embodiments of the invention (in FIG. 1a through FIG. 1f).

### DETAILED DESCRIPTION

The following description of several embodiments describes non-limiting examples that further illustrate the invention. No titles of sections contained herein, including those appearing above, are limitations on the invention, but rather they are provided to structure the illustrative description of the invention that is provided by the specification.

Unless defined otherwise, all technical and scientific terms used in this document have the same meanings that one skilled in the art to which the disclosed invention pertains would ascribe to them. The singular forms “a”, “an”, and “the” include plural referents unless the context clearly indicates otherwise. Thus, for example, reference to “fluid” refers to one or more fluids, such as two or more fluids, three or more fluids, etc. Any mention of an element includes that element's equivalents as known to those skilled in the art.

Any methods and materials similar or equivalent to those described in this document can be used in the practice or testing of the present invention. This disclosure incorporates by reference all publications mentioned in this disclosure all of the information disclosed in the publications.

This disclosure discusses publications only to facilitate describing the current invention. Their inclusion in this document is not an admission that they are effective prior art to this invention, nor does it indicate that their dates of publication or effectiveness are as printed on the document.

The features, aspects, and advantages of the invention will become more apparent from the following detailed description, appended claims, and accompanying drawings.

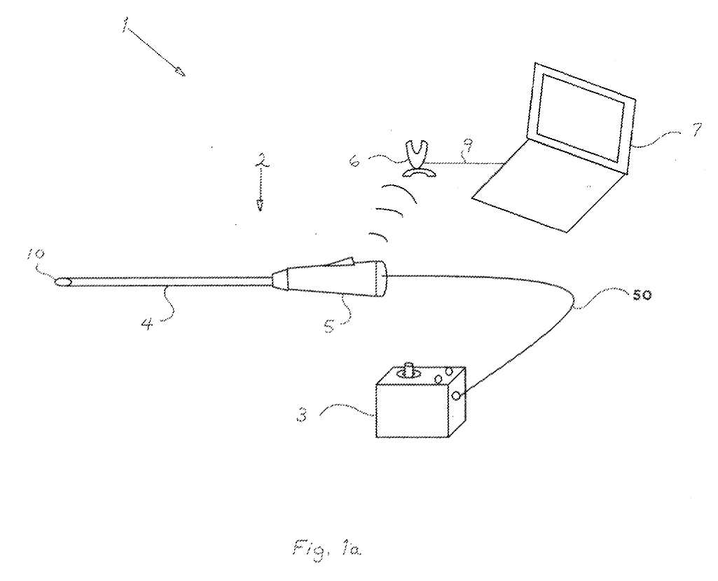

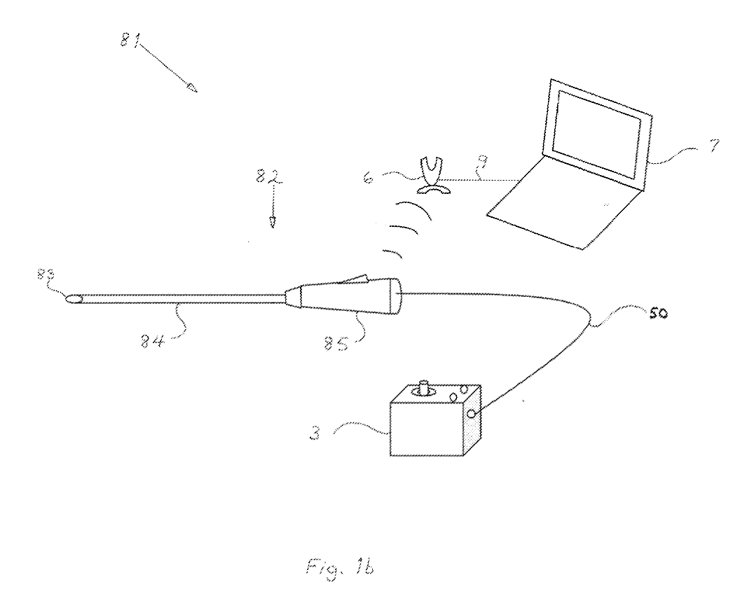

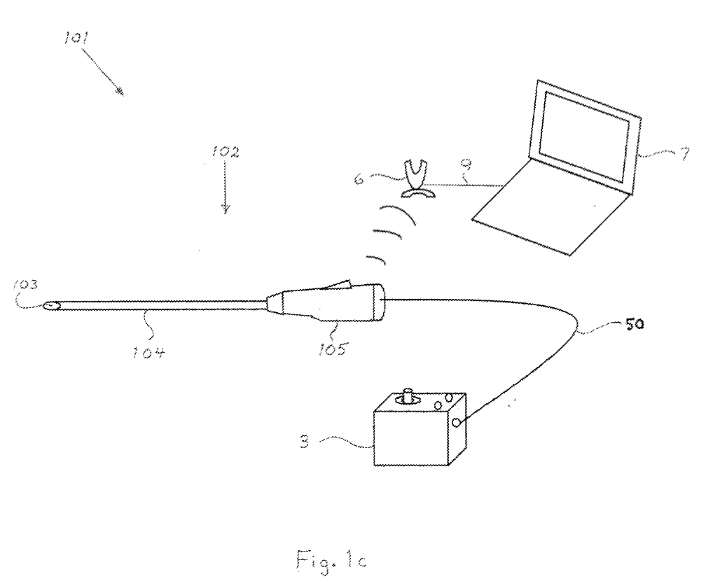

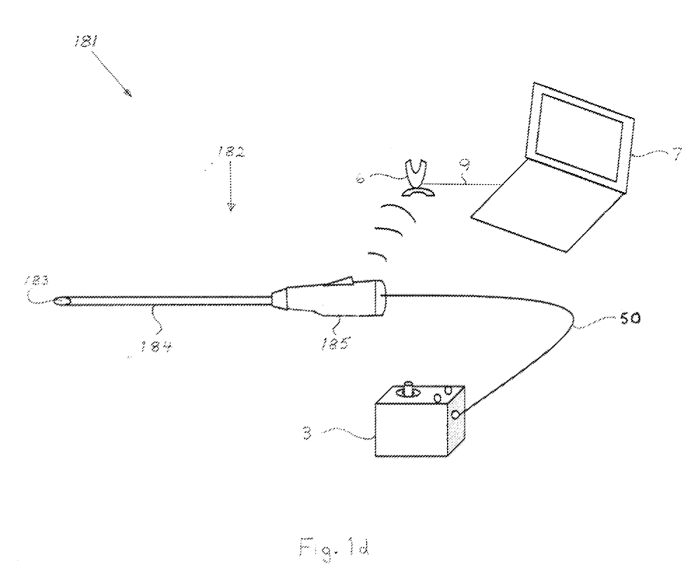

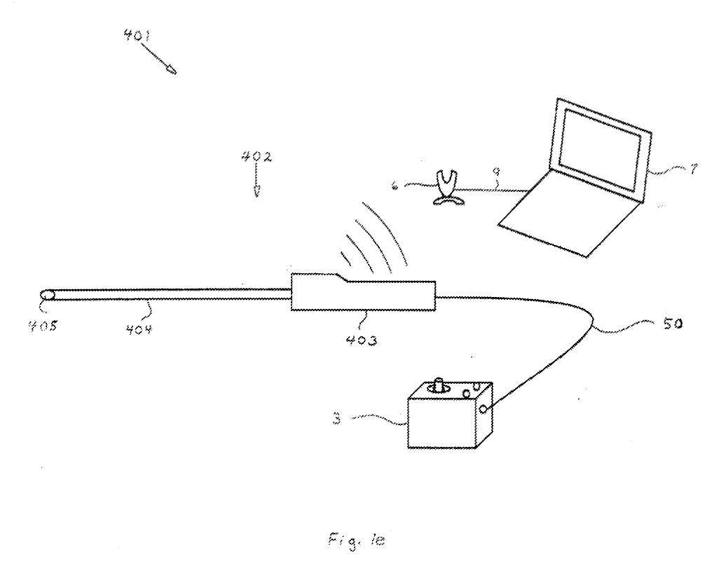

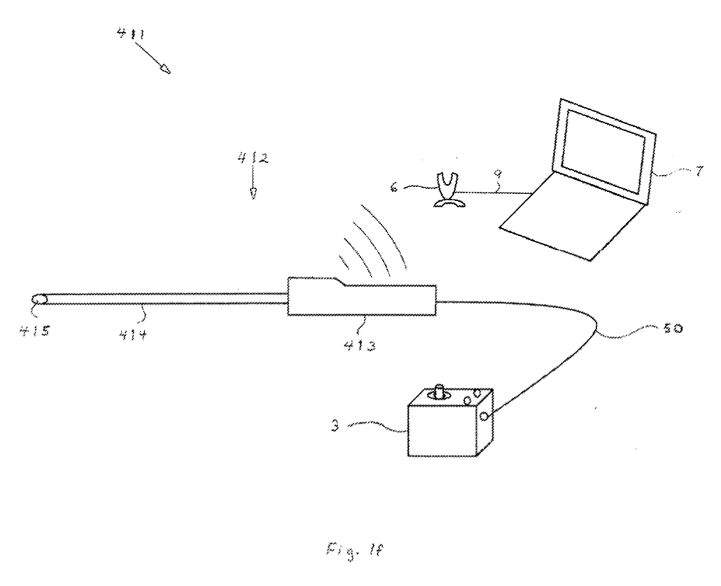

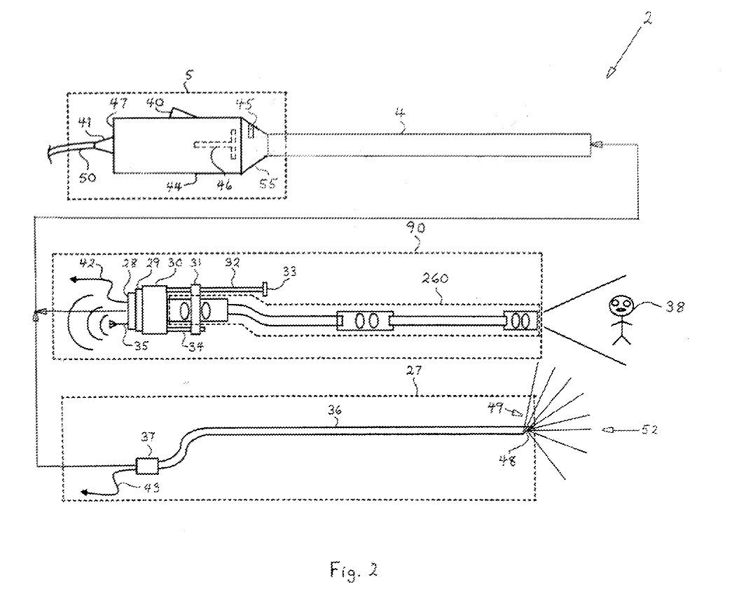

FIG. 1a shows an overall setup for a first embodiment of the invention, which is a wireless endoscope system 1. The wireless endoscope system 1 comprises an endoscopic device 2, a power and control module (PCM) 3 and a receiver 6. The endoscopic device 2 comprises an ergonomic handle 5 and a conduit 4. The conduit 4 contains an imaging system (90 in FIG. 2) that transfers an image of a target at the distal end 10 of the conduit 4 to a wireless CCD or CMOS color camera in the handle 5 of the endoscopic device 2. The wireless CCD or CMOS color camera transmits the image to the receiver 6 where the image is output 9 to a monitor 7 for display. The endoscopic device 2 also contains an illumination system (27 in FIG. 2) that provides light for the target at the distal end 10 of the conduit 4. The illumination system (27 in FIG. 2) comprises a high brightness light emitting diode (HB LED) in the handle 5 and a glass light pipe in the conduit 4 that transfers the light from the HB LED to the target. A detachable power and control module (PCM) 3 is provided to power the wireless CCD or CMOS color camera and the HB LED as well as control the light intensity of the HB LED. The electrical power for this first embodiment of the invention (FIG. 1a) is supplied by three C-cell batteries that are contained within the PCM 3. Power and control signals from the PCM 3 are connected to the endoscopic device 2 by means of cable 50. The imaging system (90 in FIG. 2), wireless CCD or CMOS color camera, and illumination system (27 in FIG. 2) are detailed in subsequent paragraphs.

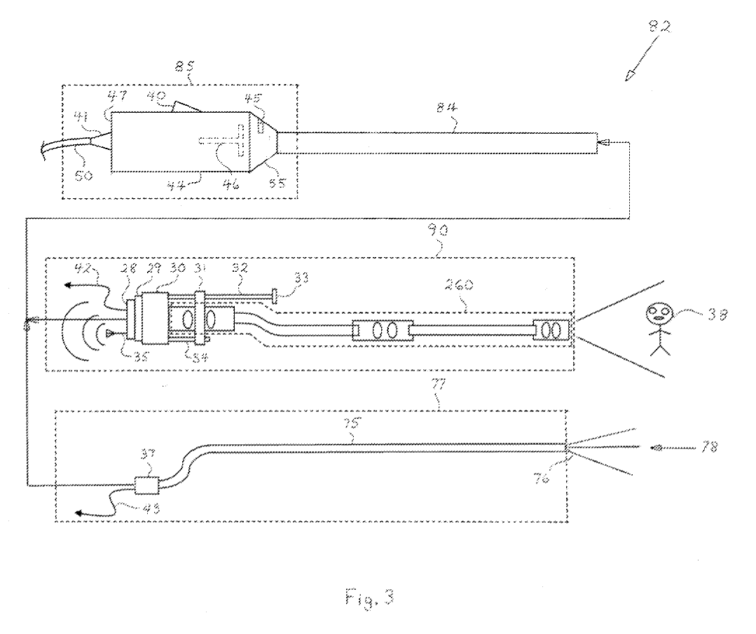

FIG. 1b shows an overall setup for a second embodiment of the invention, which is a wireless endoscope system 81. The wireless endoscope system 81 comprises an endoscopic device 82, a power and control module (PCM) 3 and a receiver 6. This second embodiment of the invention (FIG. 1b) has a handle 85, a conduit 84, an imaging system 90 of FIG. 3, and an illumination system 77 of FIG. 3. The conduit 84 has a distal end 83. All other aspects of this second embodiment of the invention (FIG. 1b) are similar to the first embodiment of the invention (FIG. 1a). The imaging system (90 in FIG. 3), wireless CCD or CMOS color camera, and illumination system (77 in FIG. 3) are detailed in subsequent paragraphs.

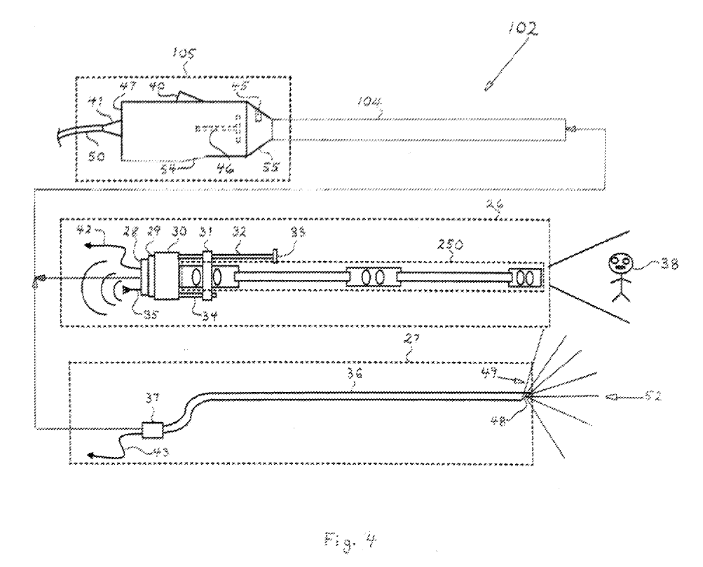

FIG. 1c shows an overall setup for a third embodiment of the invention, which is a wireless endoscope system 101. The wireless endoscope system 101 comprises an endoscopic device 102, a power and control module (PCM) 3 and a receiver 6. This third embodiment of the invention (FIG. 1c) has a handle 105, a conduit 104, an imaging system 26 of FIG. 4, and an illumination system 27 of FIG. 4. The conduit 104 has a distal end 103. All other aspects of this third embodiment of the invention (FIG. 1c) are similar to the first embodiment of the invention (FIG. 1a). The imaging system (26 in FIG. 4), wireless CCD or CMOS color camera, and illumination system (27 in FIG. 4) are detailed in subsequent paragraphs.

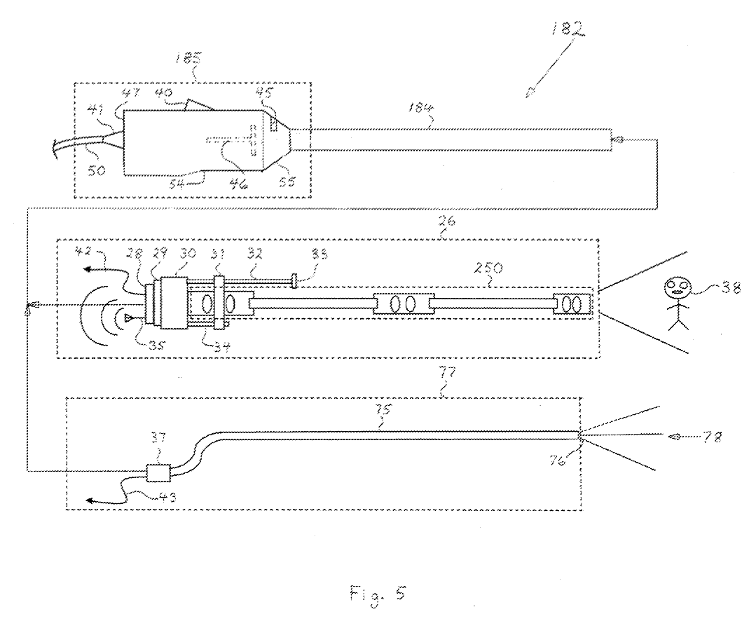

FIG. 1d shows an overall setup for a fourth embodiment of the invention, which is a wireless endoscope system 181. The wireless endoscope system 181 comprises an endoscopic device 182, a power and control module (PCM) 3 and a receiver 6. This fourth embodiment of the invention (FIG. 1d) has a handle 185, a conduit 184, an imaging system 26 of FIG. 5, and an illumination system 77 of FIG. 5. The conduit 184 has a distal end 183. All other aspects of this fourth embodiment of the invention (FIG. 1d) are similar to the first embodiment of the invention (FIG. 1a). The imaging system (26 in FIG. 5), wireless CCD or CMOS color camera, and illumination system (77 in FIG. 5) are detailed in subsequent paragraphs.

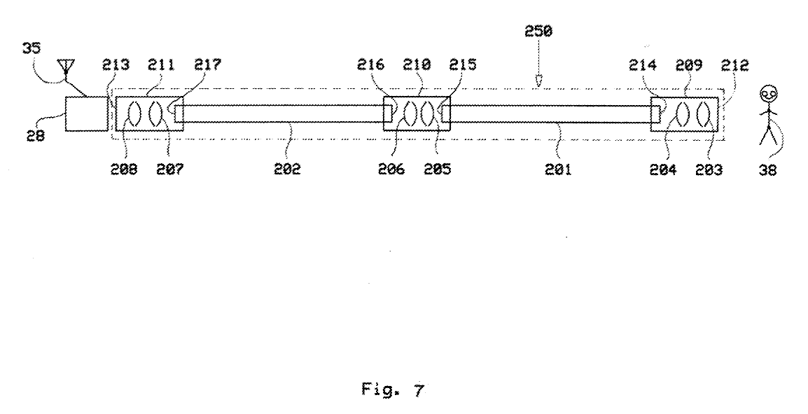

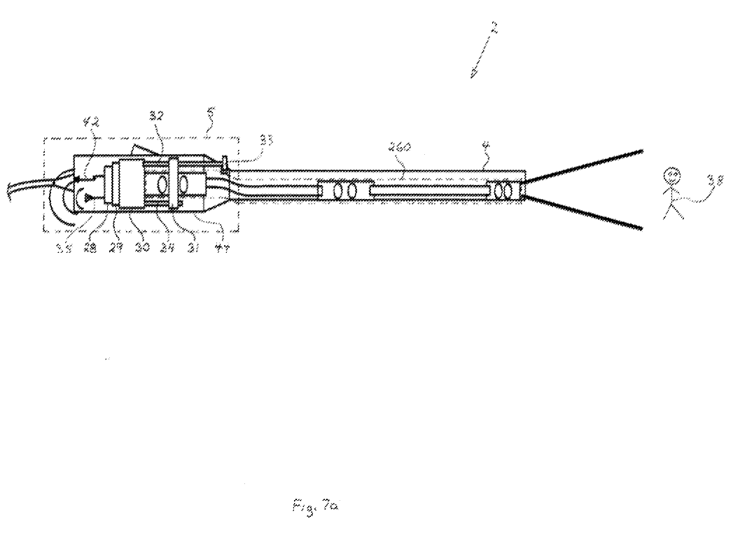

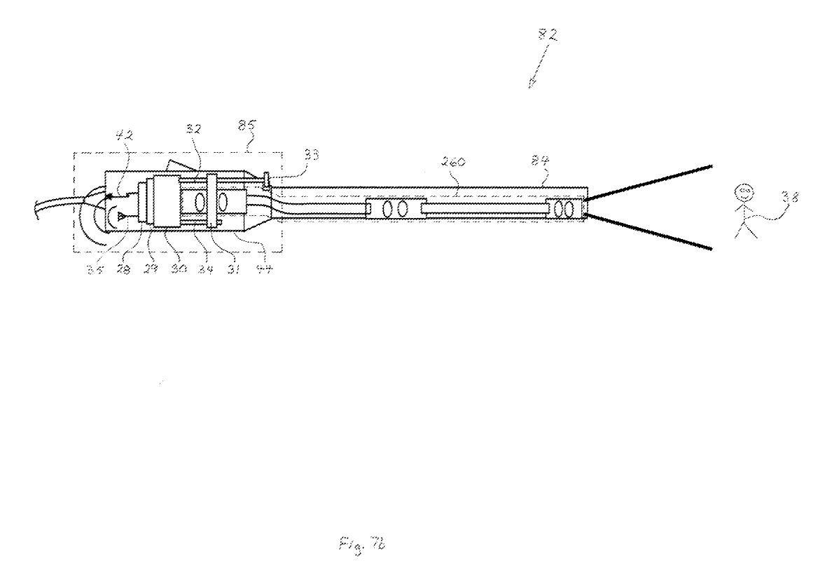

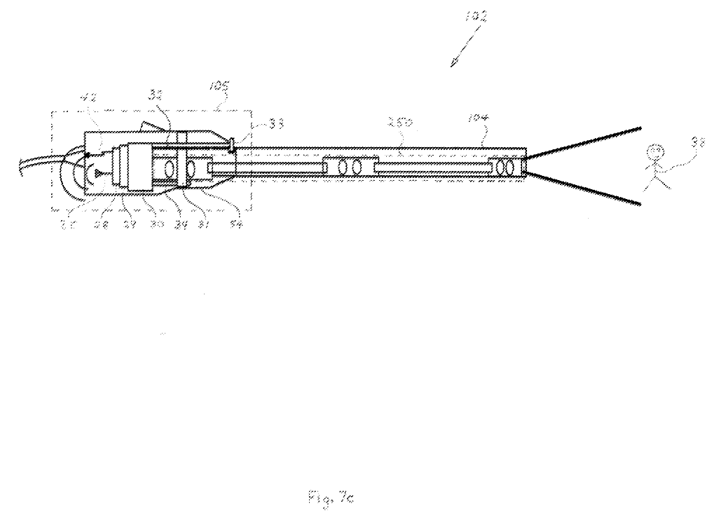

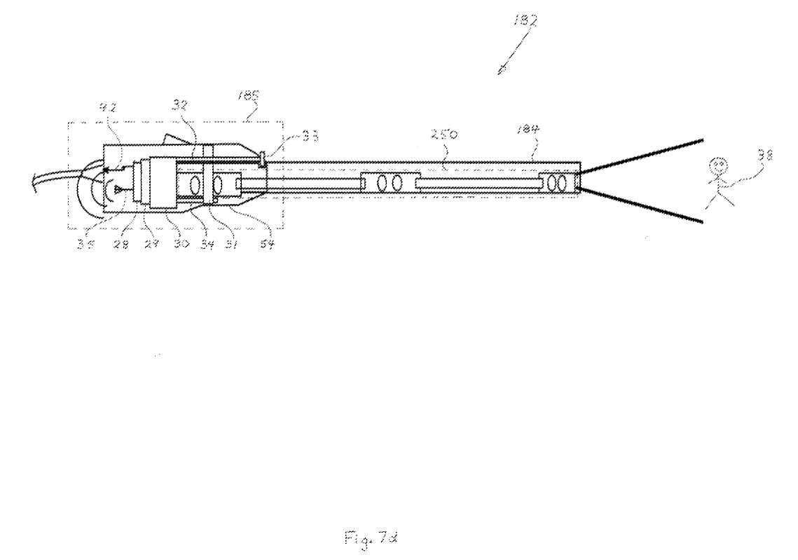

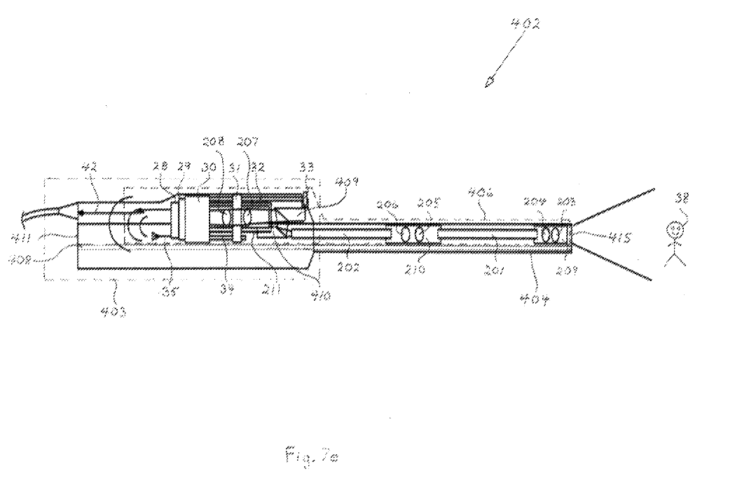

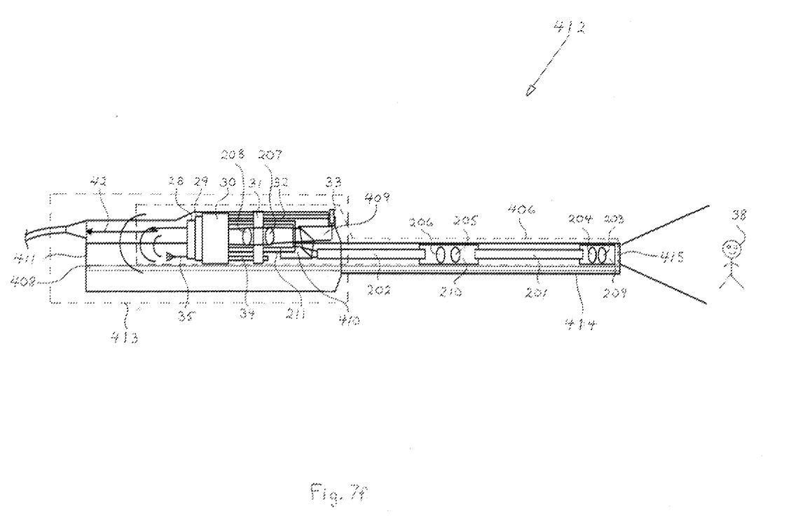

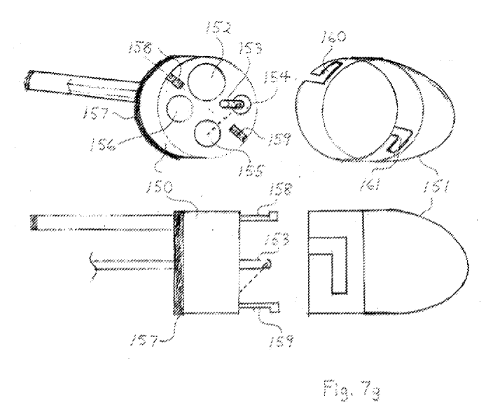

FIG. 1e shows an overall setup for the fifth embodiment of the invention, which is a wireless endoscope system 401. The wireless endoscope system 401 comprises an endoscopic device 402, a power and control module (PCM) 3 and a receiver 6. This fifth embodiment of the invention (FIG. 1e) has a handle 403, a conduit 404, an imaging system 406 of FIG. 7e and an illumination system 27 of FIG. 2 and FIG. 4. The conduit 404 has a distal end 405. All other aspects of this fifth embodiment of the invention (FIG. 1e) are similar to the first embodiment of the invention detailed in FIG. 1a. The imaging system (406 of FIG. 7e), wireless CCD or CMOS color camera, and illumination system (27 of FIG. 2 and FIG. 4) are detailed in subsequent paragraphs.

FIG. 1f shows an overall setup for the sixth embodiment of the invention, which is a wireless endoscope system 411. The wireless endoscope system 411 comprises an endoscopic device 412, a power and control module (PCM) 3 and a receiver 6. This sixth embodiment of the invention (FIG. 1f) has a handle 413, a conduit 414, an imaging system 406 of FIG. 7f and an illumination system 77 of FIG. 3 and FIG. 5. The conduit 414 has a distal end 415. All other aspects of this sixth embodiment of the invention (FIG. 1f) are similar to the first embodiment of the invention detailed in FIG. 1a. The imaging system (406 of FIG. 7f), wireless CCD or CMOS color camera, and illumination system (77 of FIG. 3 and FIG. 5) are detailed in subsequent paragraphs.

### Description of the Wireless CCD or CMOS Color Camera:

Referring to FIG. 2; in this first embodiment of the endoscopic device 2, the camera 28 is a wireless CCD or CMOS color camera whose output images are transmitted by a high-frequency transmitter circuit operating in the Industrial, Scientific and Medical (ISM) frequency band. In the first embodiment (FIG. 2) of the endoscopic device 2, the ISM frequency chosen is in the 2.4 GHz frequency band. Other possible frequencies that can be used in other embodiments are in the 5.8 GHz frequency band, or in the 900 MHz frequency band, or in the 2.36 GHz to 2.39 GHz band known as the Wireless Body Area Network, or WBAN. In some embodiments, wireless CCD or CMOS color camera 28 is housed inside the handle 5 of the endoscopic device 2 and is powered from the power and control module 3 of FIG. 1a through FIG. 1f. The above details regarding the wireless CCD or CMOS color camera 28, also apply to the other five embodiments of the endoscopic devices: see 28 in FIG. 3 through FIG. 5 as well as 28 in FIG. 7e and FIG. 7f. While this disclosure refers to CCD or CMOS cameras, any type of camera detector can be useful in cameras used in this invention.

### Descriptions of Wireless Endoscope System Embodiments:

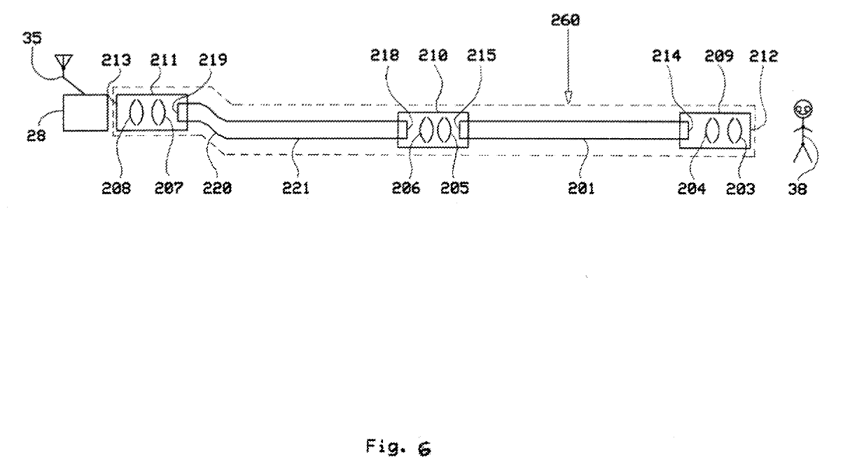

FIG. 2 details the first embodiment of the endoscopic device 2 (see also 2 in FIG. 1a) which comprises a handle 5, a conduit 4, a first embodiment of the imaging system 90 and a first embodiment of the illumination system 27. The imaging system 90 further comprises the imaging assembly 260, a wireless CCD or CMOS color camera 28, and a coupler component 29 that interfaces the wireless CCD or CMOS color camera 28 to the image focusing mechanism. As seen in FIG. 2, one of the components of the imaging assembly 260 has an s-bend in it (see FIG. 6 description for further details). The image focusing mechanism comprises components 30 through 34 and has the function of focusing an image of the target 38 as it appears at the proximal end (213 of FIG. 6) of the imaging assembly 260 onto the CCD or CMOS element of the wireless color camera 28. The focusing mechanism (30 through 34), the coupler component 29, and the wireless CCD or CMOS color camera 28 are contained within the handle 5. The main components of the handle 5 are the handle body 44, the handle end cap 47, and a cutout 45 in the distal end 55 of the handle 5 for the focusing thumb wheel 33 and a T-slot cutout 46 for a manipulation tool (not shown). The transmitting antenna 35 of the wireless CCD or CMOS color camera 28 is also contained within the handle 5. Power for the wireless CCD or CMOS color camera 28 is provided by the power and control module (PCM) 3 (of FIG. 1a through 1f) and is applied by means of the wiring 42 and cable 50. In some embodiments, the light source required to illuminate the target 38 at the distal end of the imaging assembly 260 is provided by a single high brightness light emitting diode (HB LED) 37 that is housed in the handle 5. The light from the HB LED 37 is transmitted to the target 38 through a glass light pipe 36. In this first embodiment of the endoscopic device 2, the distal end 48 of the light pipe 36 has been cut and polished to a 30 degree angle (+/−5 degrees) to form a Non-Imaging Optic Tip 48. (For purposes of this disclosure, the angle is measured relative to the axis that runs parallel to the long axis of light pipe 36. An angle of 90 degrees indicates an angle perpendicular to the long axis; an angle of 30 degrees indicates an angle 30 degrees counterclockwise from the axis, in the quadrant between zero degrees from the axis and perpendicular to the axis.). A purpose of the Non-Imaging Optic Tip 48 is to produce light for the target 38 from both the outside as well as the inside 49 (side-firing) of the tip 48 producing a wide light pattern 52 on the target 38. Power and an intensity control signal for the HB LED 37 are provided by the PCM 3 (in FIG. 1a through 1f), are applied through the wiring 43, and cable 50. A strain relief 41 provides a flexible anchor for the cable 50 as it enters the handle 5. The imaging assembly 260 and the light pipe 36 are contained within separate lumens (not shown in FIG. 2) that are contained within the conduit 4. Another lumen in the conduit 4 provides a path to the distal end of the conduit for a surgical tool entered at the surgical tool port 40 on the handle 5. But there are other embodiments in which the imaging assembly 260 and the light pipe 36 are contained within the same conduit 4.

FIG. 3 details a second embodiment of the endoscopic device 82, and one difference is that the endoscopic device 82 further contains a second embodiment of the illumination system 77. In this illumination system 77, the tip 76 of the light pipe 75 has been polished flat at 90 degrees to the light pipe 75 producing a narrower light pattern 78 for the target 38. Even as some of the numbers on the FIG. 3 drawing have changed from the FIG. 2 drawing, the functions of the respective components in FIG. 3 are similar to those detailed in FIG. 2.

FIG. 4 details a third embodiment of the endoscopic device 102, and one difference is that the endoscopic device 102 further contains a second embodiment of the imaging system 26. As seen in FIG. 4, the components of the imaging assembly 250 are in a straight-line configuration (see FIG. 7 description for further details). This change to the imaging assembly 250 is facilitated by the handle 105 of the endoscopic device 102 having a slightly larger diameter handle body 54. Even as some of the numbers on the FIG. 4 drawing have changed from the FIG. 2 drawing, the functions of the respective components in FIG. 4 are similar to those detailed in FIG. 2.

FIG. 5 details a fourth embodiment of the endoscopic device 182. This embodiment of the endoscopic device 182 combines elements of the second embodiment of the imaging system 26 and the second embodiment of the illumination system 77 that has the flat face 76 of the light pipe 75. This change to the imaging assembly 250 is facilitated by the handle 185 of the endoscopic device 182 having a slightly larger diameter handle body 54. Even as some of the numbers on the FIG. 5 drawing have changed from the FIG. 2 drawing, the functions of the respective components in FIG. 5 are similar to those detailed in FIG. 2.

### Descriptions of the Imaging Assembly Embodiments:

FIG. 6 details a first embodiment of an imaging assembly 260 that is part of the first embodiment of the endoscopic device 2 in FIG. 2 and the second embodiment of the endoscopic device 82 in FIG. 3. This imaging assembly 260 comprises two segmented coherent fiber (CF) bundles 201 and 221, six achromatic optic elements 203 through 208, and three dual-lens housings 209, 210, and 211. The two segmented CF bundles (201 and 221) comprise fiber segments of a length and diameter appropriate to fit the endoscopic devices 2 in FIG. 2 and 82 in FIG. 3. A purpose of the two segmented CF bundles (201 and 221) is to relay an image of the target 38 through close-packed fibers while keeping the same orientation of the image of the target 38 that was first formed on the face 214 of the first segmented CF bundle 201. Achromatic optic elements 203 through 208 are employed to transfer and focus an image of the target 38 being viewed at the distal end 212 of the imaging assembly 260. Each of the optic elements (203 through 208) is made with different glass and grind radius in order to account for spherical and chromatic aberrations of an image being viewed. The first two optic elements (203 and 204) are contained within the first dual-lens housing 209 at the distal end 212 of the imaging assembly 260. A purpose of these two optical elements (203 and 204) is to collect and transfer an image of the target 38 to the distal face 214 of the first segmented CF bundle 201. The second two optic elements 205 and 206 are contained in the second dual-lens housing 210. These two optic elements (205 and 206) have focal lengths that facilitate the image at the proximal end 215 of the first segmented CF bundle 201 being transferred to the distal end 218 of the second segmented CF bundle 221 without substantial distortion. This technique is known as Free Space Optical Coupling. A purpose of the third set of optic elements 207 and 208 and the dual-lens housing 211 is similar to the optic elements contained in dual-lens housings 209 and 210. However, the magnification levels of optic elements 207 and 208 can be changed in order to adjust the size of the image as it is viewed on a video monitor (7 in FIG. 1a through FIG. 1f). The image at the proximal end 213 of the imaging assembly 260 is coupled (29 in FIG. 7a through FIG. 7f) to the CCD or CMOS element of a wireless CCD or CMOS color camera (28 in FIG. 7a through FIG. 7f) where it is transmitted by means of an antenna (35 in FIG. 7a through FIG. 7f) to a compatible receiver (6 in FIG. 1a through FIG. 1f). A difference between imaging assembly 250 and imaging assembly 260 is that the segmented CF bundle 221 has an s-bend 220 towards the proximal end of the bundle 221. This change to the imaging assembly 260 allows the handles 5 in FIG. 2 and 85 in FIG. 3 of the invention, to have a smaller diameter.

FIG. 7 details a second embodiment of an imaging assembly 250 that is part of the third embodiment of the endoscopic device 102 in FIG. 4 and the fourth embodiment of the endoscopic device 182 in FIG. 5. This imaging assembly 250 comprises two segmented coherent fiber (CF) bundles 201 and 202, six achromatic optic elements 203 through 208, and three dual-lens housings 209, 210, and 211. The two segmented CF bundles (201 and 202) are identical and comprise fiber segments of a length and diameter appropriate to fit the endoscopic devices 102 in FIG. 4 and 182 in FIG. 5. A purpose of the two segmented CF bundles (201 and 202) is to relay an image of the target 38 through close-packed fibers while keeping the same orientation of the image of the target 38 that was first formed on the face 214 of the first segmented CF bundle 201. Achromatic optic elements 203 through 208 are employed to transfer and focus an image of the target 38 being viewed at the distal end 212 of the imaging assembly 250. Each of the optic elements (203 through 208) is made with different glass and grind radius in order to account for spherical and chromatic aberrations of an image being viewed. The first two optic elements (203 and 204) are contained within the first dual-lens housing 209 at the distal end 212 of the imaging assembly 250. A purpose of these two optical elements (203 and 204) is to collect and transfer an image of the target 38 to the distal face 214 of the first segmented CF bundle 201. The second two optic elements 205 and 206 are contained in the second dual-lens housing 210. These two optic elements (205 and 206) have focal lengths that facilitate the image at the proximal end 215 of the first segmented CF bundle 201 being transferred to the distal end 216 of the second segmented CF bundle 202 without substantial distortion. This technique is known as Free Space Optical Coupling. A purpose of the third set of optic elements 207 and 208 and the dual-lens housing 211 is similar to the optic elements contained in dual-lens housings 209 and 210. However, the magnification levels of optic elements 207 and 208 can be changed in order to adjust the size of the image as it is viewed on a video monitor (7 in FIG. 1a through FIG. 1f). The image at the proximal end 213 of the imaging assembly 250 is coupled (29 in FIG. 7a through FIG. 7f) to the CCD or CMOS element of a wireless CCD or CMOS color camera (28 in FIG. 7a through FIG. 7f) where it is transmitted by means of an antenna (35 in FIG. 7a through FIG. 7f) to a compatible receiver (6 in FIG. 1a through FIG. 1f).

### Descriptions of Partial Assembly of Endoscopic Device Embodiments:

FIG. 7a details a partial assembly of the first embodiment of the endoscopic device 2 (see also 2 in FIG. 1a). A purpose of FIG. 7a is to show how the first embodiment of the imaging system 90 of FIG. 2 fits within the handle 5 and the conduit 4 of the endoscopic device 2. Included in the first embodiment of the imaging system (90 of FIG. 2) are the imaging assembly 260, the wireless CCD or CMOS color camera 28, the camera power connection 42, the camera antenna 35, the coupler component 29, and the focusing mechanism components 30 through 34. The s-bend in the imaging assembly 260 allows for a simpler handle body 44 but retains the imaging assembly 260 offset in the conduit 4 to accommodate other components such as the first embodiment of the illumination system 27 of FIG. 2 that also reside in the handle 5 and the conduit 4 of the endoscopic device 2. The imaging assembly 260 offset also facilitates accommodating a surgical tool lumen along the top of the inside of the conduit 104. Other components have been omitted in FIG. 7a for clarity.

FIG. 7b details a partial assembly of the second embodiment of the endoscopic device 82 (see also 82 in FIG. 1b). A purpose of FIG. 7b is to show how the first embodiment of the imaging system 90 of FIG. 3 fits within the handle 85 and the conduit 84 of the endoscopic device 82. Included in the first embodiment of the imaging system (90 of FIG. 2) are the imaging assembly 260, the wireless CCD or CMOS color camera 28, the camera power connection 42, the camera antenna 35, the coupler component 29, and the focusing mechanism components 30 through 34. The s-bend in the imaging assembly 260 allows for a simpler handle body 44 but retains the imaging assembly 260 offset in the conduit 84 to accommodate other components such as the second embodiment of the illumination system 77 of FIG. 3 that also reside in the handle 85 and the conduit 84 of the endoscopic device 82. The imaging assembly 260 offset also facilitates accommodating a surgical tool lumen along the top of the inside of the conduit 104. Other components have been omitted in FIG. 7b for clarity.

FIG. 7c details a partial assembly of the third embodiment of the endoscopic device 102 (see also 102 in FIG. 1c). A purpose of FIG. 7c is to show how the second embodiment of the imaging system 26 of FIG. 4 fits within the handle 105 and the conduit 104 of the endoscopic device 102. Included in this second embodiment of the imaging system (26 of FIG. 4) are the imaging assembly 250, the wireless CCD or CMOS color camera 28, the camera power connection 42, the camera antenna 35, the coupler component 29, and the focusing mechanism components 30 through 34. The handle body 54 has changed shape to facilitate the offset of the imaging assembly 250 within the conduit 104 of the endoscopic device 102. Imaging assembly 250 is offset in the conduit 104 to accommodate other components such as the first embodiment of the illumination system 27 of FIG. 4 that also reside in the handle 105 and the conduit 104 of the endoscopic device 102. The imaging assembly offset also facilitates accommodating a surgical tool lumen along the top of the inside of the conduit 104. Other components have been omitted in FIG. 7c for clarity.

FIG. 7d details a partial assembly of the fourth embodiment of the endoscopic device 182 (see also 182 in FIG. 1d). A purpose of FIG. 7d is to show how the second embodiment of the imaging system 26 of FIG. 5 fits within the handle 185 and the conduit 184 of the endoscopic device 182. Included in this second embodiment of the imaging system (26 of FIG. 5) are the imaging assembly 250, the wireless CCD or CMOS color camera 28, the camera power connection 42, the camera antenna 35, the coupler component 29, and the focusing mechanism components 30 through 34. The handle body 54 has changed shape to facilitate the offset of the imaging assembly 250 within the conduit 184 of the endoscopic device 182. Imaging assembly 250 is offset in the conduit 184 to accommodate other components such as the second embodiment of the illumination system 77 of FIG. 5 that also reside in the handle 185 and the conduit 184 of the endoscopic device 182. The imaging assembly offset also facilitates accommodating a surgical tool lumen along the top of the inside of the conduit 184. Other components have been omitted in FIG. 7d for clarity.

FIG. 7e details a partial assembly of the fifth embodiment of the endoscopic device 402 (see also 402 in FIG. 1e). A purpose of FIG. 7e is to show how the first embodiment of the dual-folded imaging system 406 fits within the handle 403 and conduit 404 of the endoscopic device 402. The position of imaging system 406 within the endoscopic device 402 is important because it facilitates a horizontal lumen 408 passing from the proximal end 411 of the endoscopic device 402 to the distal end 415 of the endoscopic device 402. This lumen 408 provides a low-friction path through the handle 403 and conduit 404 for a surgical device inserted at the proximal end 411 of the endoscopic device 402. In order to accommodate this horizontal lumen 408, the wireless CCD or CMOS color camera 28, the coupler component 29, the focusing mechanism components (30 through 34) and one of the dual-lens housing 211 have been shifted off center of the handle 403. The two 45 degree mirrors 409 and 410, prevent substantial degradation of an image of the target 38 that sometimes occurs with an abrupt bend in the second coherent fiber (CF) bundle 202. This embodiment of the endoscopic device 402 also contains elements of the first embodiment of the illumination system 27 of FIG. 2 and FIG. 4.

FIG. 7f details a partial assembly of the sixth embodiment of the endoscopic device 412 (see also 412 in FIG. 1f). A purpose of FIG. 7f is to show how the first embodiment of the dual-folded imaging system 406 fits within the handle 413 and conduit 414 of the endoscopic device 412. The position of imaging system 406 within the endoscopic device 412 is important because it allows a horizontal lumen 408 to pass from the proximal end 411 of the endoscopic device 412 to the distal end 415 of the endoscopic device 412. This lumen 408 provides a low-friction path through the handle 413 and conduit 414 for a surgical device inserted at the proximal end 411 of the endoscopic device 412. In order to accommodate this horizontal lumen 408, the wireless CCD or CMOS color camera 28, the coupler component 29, the focusing mechanism components (30 through 34), and one of the dual-lens housing 211 have been shifted off center of the handle 413. The two 45 degree mirrors 409 and 410 prevent substantial degradation of an image of the target 38 that could otherwise occur with an abrupt bend in the second coherent fiber (CF) bundle 202. This embodiment of the endoscopic device 412 also contains elements of the second embodiment of the illumination system 77 of FIG. 3 and FIG. 5.

### Description of the Distal Baffle and the Optically Transparent Shield:

FIG. 7g provides two views of a distal lumen baffle 150 that is placed at the distal end of each of the six embodiments of the endoscopic devices (2 in FIG. 1a, 82 in FIG. 1b, 102 in FIG. 1c, 182 in FIG. 1d, 402 in FIG. 1e and 412 in FIG. 1f). Cutouts 152, 154, 155 and 156 in the baffle 150 are for various lumens that are contained within the endoscopic devices. The baffle 150 is secured at the distal end of each of the endoscopic devices by the conduit seal 157. The distal end of a lens washing system 153 is shown along with two locking pins 158 and 159 that mate with locking slots 160 and 161 to secure the optically transparent shield 151 against the baffle 150 when the shield 151 is required during a surgical procedure. Of course, one of ordinary skill in the art will recognize that other embodiments exist that use a structure differing from that of distal lumen baffle 150 to provide functionality similar to that of the baffle.

### Description of the Power & Control System:

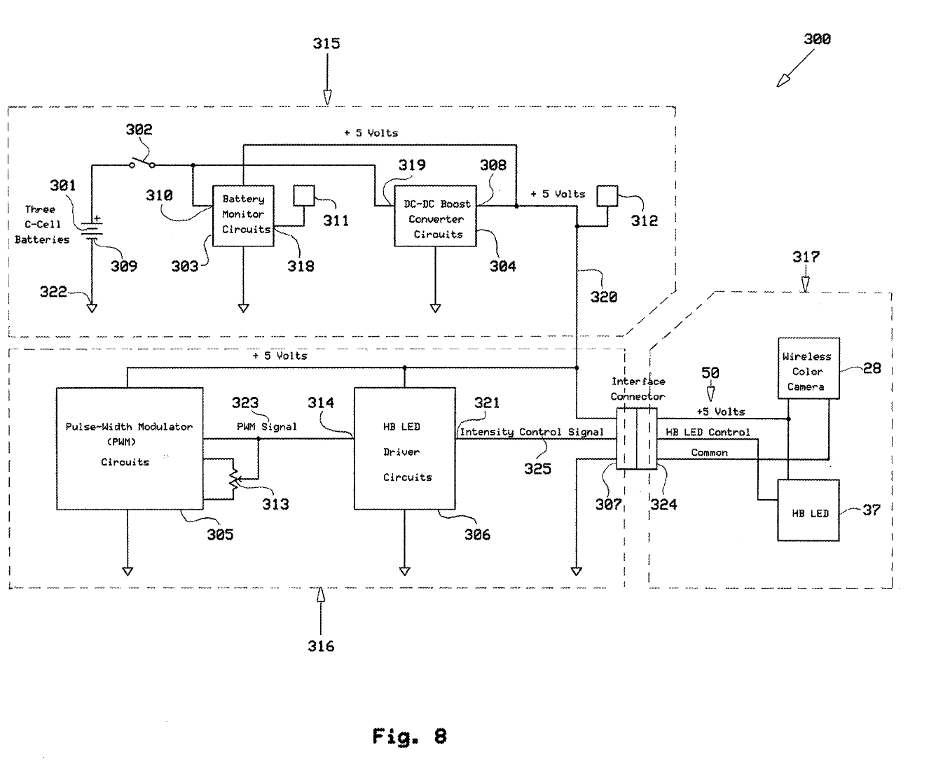

FIG. 8 depicts a wiring diagram 300 of the power and control module (PCM) 3 (in FIG. 1a, through FIG. 1f) for six embodiments of the invention. Since a purpose of the PCM is to provide power and intensity control to a high brightness light emitting diode (HB LED) 37 and power to a wireless CCD or CMOS color camera 28, these components are included on this wiring diagram 300 for reference only and have been described in detail in earlier sections of this document.

FIG. 8 presents the electrical system 315 for the invention that comprises an energy source 301, battery monitor circuits 303 and voltage converter circuits 304. The energy source in this embodiment of the invention is three standard C-cell batteries 301. The batteries 301 provide power to the voltage converter circuits 304 that produces a regulated voltage for the remainder of the electronic circuits and components of various invention embodiments. The schematic symbol for ground 322 (or common circuit) connects the negative side 309 of the batteries 301 to all of the electronic circuits and components of various invention embodiments. A battery monitor circuit 303 provides a means of indicating when the battery voltage has reached a preset low level. The battery monitor input 310 is connected to the batteries 301 through the switch 302. An indicator device 311 (yellow LED in this embodiment of the invention) is connected to the battery monitor output 318 and will present a visual indication (yellow LED lights in this embodiment of the invention) when the battery voltage has reached a voltage equal to or less than a preset low level. An ON-OFF switch 302 is provided so that the batteries 301 can be connected to or disconnected from the electrical system 315. The main component of the voltage converter circuits 304 is a DC-to-DC boost voltage converter integrated circuit. When the batteries 301 are connected via the switch 302 to the voltage converter input 319, the voltage converter circuits produce a constant voltage at output 308. Even as the battery voltage begins to drop with use, or as the current drawn from the voltage converter circuits 304 varies widely, the voltage at the output 308 of the voltage converter circuits 304 will remain constant. Whenever switch 302 is closed and batteries 301 are in place, a second indicator device 312 (green LED in this embodiment of the invention), attached to the regulated voltage circuit 320, will present a visual indication (green LED lights in this embodiment of the invention) that the electrical system 315 of FIG. 8 has been powered up. The regulated voltage at 320 is supplied to the wireless CCD or CMOS color camera 28 and high brightness light emitting diode (HB LED) 37 by means of connectors 307 and 324 and a cable 50. Of course, one of ordinary skill in the art will recognize that other embodiments exist that use other types of batteries of that use a power source other than batteries, such as a wall outlet, capacitor-based energy source, or other power source invented in the future.

### Description of the HB LED Intensity Control System:

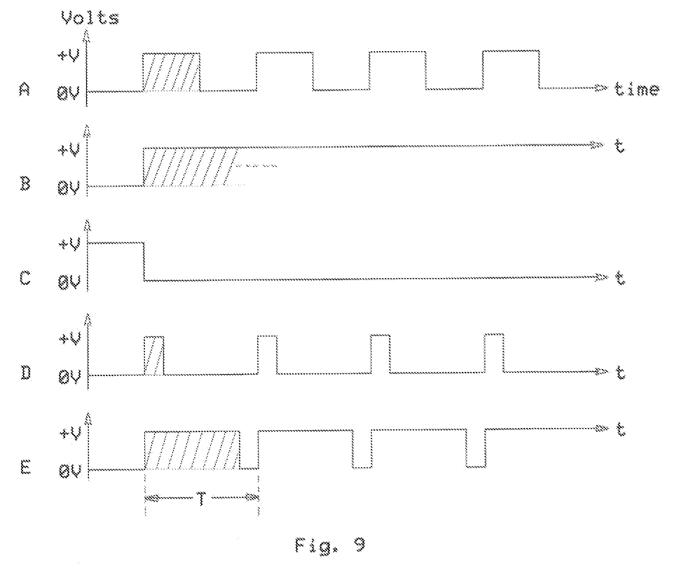

Referring to FIG. 8; in some embodiments of the invention, a high brightness light emitting diode (HB LED) 37, housed in the handles (5 in FIG. 1a, 85 in FIG. 1b, 105 in FIG. 1c, 185 in FIG. 1d, 403 in FIG. 1e and 413 in FIG. 1f) of the invention, provides varying intensity white light to the target 38 (in FIG. 2 through FIG. 5, FIG. 7e and FIG. 7f). The intensity of the light produced by the HB LED 37 can be varied by a pulse-width modulated (PWM) intensity control signal 325 generated by the PWM system 316. The PWM system 316 comprises pulse-width modulator circuits 305 and HB LED driver circuits 306. The PWM circuits 305 produce a fixed-frequency square-wave where the width of half of one full cycle of the square-wave (see ‘T’ in FIG. 9) is varied by potentiometer 313. The PWM signal 323 produced by the PWM circuits 305, is then fed to the input 314 of the HB LED driver circuits 306. The HB LED driver circuits 306 converts the PWM signal at input 314 to a PWM intensity control signal 325 that has sufficient power to light the HB LED 37. This signal at the output 321 of the HB LED driver circuits 306 is fed to the HB LED 37 by means of connectors 307 and 324, and cable 50. Various light intensities for the HB LED 37 can be produced from the example waveform diagrams of FIG. 9, A through E. These waveforms (FIG. 9, A through E) indicate that as the pulse-width varies, while the cycle time ‘T’ remains constant, the total power applied to the HB LED 37 (shaded areas of waveforms) will vary, thus increasing or decreasing the intensity of the HB LED 37.

### Description of the Interface Connectors and Cable:

Referring again to FIG. 8, the interface connectors 307 and 324 are two separate components; a panel-mount type connector 307 and a mating inline connector 324 that is attached to cable 50. The connectors 307 and 324 each have two separate contacts and a common ground; the cable 50 is a small diameter, flexible, three-wire cable. Connector 324 is permanently wired to one end of the cable 50 while the other end of the cable 50 is wired to the wireless CCD or CMOS color camera 28 and HB LED 37 in the handle sections of the invention.

### Description of the HB LED Intensity Control Waveforms:

Referring to FIG. 9; it has been stated that the pulse-width modulated (PWM) signal at the input 314 (of FIG. 8) of the HB LED Driver circuits 306 (of FIG. 8) is the result of varying the resistance of potentiometer 313 (of FIG. 8). The shapes of possible PWM signals from this action are shown in FIG. 9, A through E. It can be demonstrated that for each of the waveforms (A through E) shown in FIG. 9, the intensity of the HB LED 37 (of FIG. 8) would vary widely. For example, for the waveform at A, the intensity of the HB LED 37 (of FIG. 8) would be expected to be at half or near half of its brightest setting since power to the HB LED would only be applied for one half of one cycle time ‘T’ as indicated by the shaded portion of the waveform. Variations in intensity of the HB LED 37 (of FIG. 8), above and below this half setting, would be expected from the waveforms of B through E given that the shaded portions of the waveforms indicate power to the HB LED. Note that waveforms B and C represent conditions where the HB LED 37 (of FIG. 8) would be at full intensity or completely OFF, respectively.

### Description of the ABS Enclosure for Electrical System:

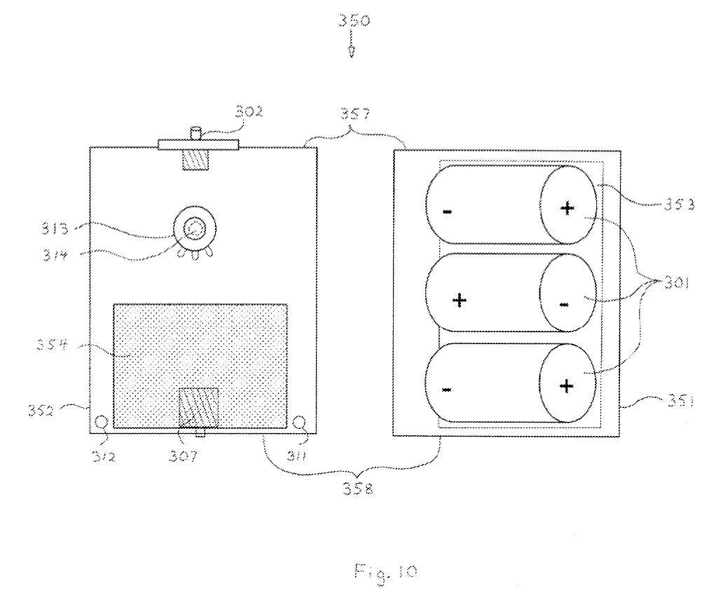

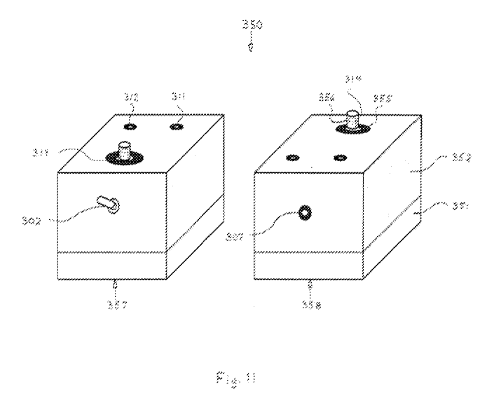

Referring to FIG. 10; in some embodiments of the invention, the electrical system 315 (of FIG. 8) and the PWM power system 316 (of FIG. 8), are housed in a small plastic enclosure 350. The enclosure 350 is composed of at least two parts: a bottom section 351 and a top section 352. A battery holder 353, in some embodiments, designed to hold three C-cell batteries, is part of the bottom section 351 of the enclosure 350. A printed circuit board (PCB) 354 is attached to the inside of the top section 352 of the enclosure 350. The PCB 354 holds most of the electronic components of the electrical system 315 (of FIG. 8) and the PWM power system 316 (of FIG. 8). Connector 307 is attached to the PCB and protrudes through a hole cut in the end of the enclosure 350. Connector 307 feeds power to the wireless CCD or CMOS color camera 28 (of FIG. 8) and power and intensity control to the HB LED 37 (of FIG. 8). Some electronic components that are not a part of the PCB 354 are the switch 302, the indicators 311 and 312, and the potentiometer 313. These four components (302, 311, 312, and 313) are attached at various locations to the top section 352 of the enclosure 350 and the components are wired to the PCB 354. The shaft 314 of potentiometer 313 protrudes through the top 352 of enclosure 350 to allow for the attachment of the dial plate 355 (of FIG. 11) and an indicator knob 356 (of FIG. 11) on the outside of the enclosure 350. This embodiment of the enclosure 350 is designed to sit on a table or tray. In another embodiment of the design, a loop or hook could be attached to the outside of the bottom section 351 of the enclosure 350 so that the power and control module (PCM) 3 (in FIG. 1a through FIG. 1f) could be worn on the hip of the operator of the invention.

Referring to FIG. 11; since the potentiometer 313 (of FIG. 10) is used to vary the intensity of the HB LED 37 (in FIG. 8), there should be some indicator of the relative position of the potentiometer so that the position would provide an indication of the intensity of the light from the HB LED 37 (of FIG. 8). A dial plate 355 and an indicator knob 356, attached to the shaft 314 of the potentiometer 313 (of FIG. 10) on the outside of the top section 352 of the enclosure 350, provide the necessary indicating mechanism. Also indicated in FIG. 11 are two sections of the enclosure: top section 352 and bottom section 351. The two indicator LEDs 311 and 312 are shown attached to the top 352 section of the enclosure 350 as are the switch 302 and the connector 307 shown at each of the ends 357 and 358 of the enclosure 350.

Returning to endoscopic device 2 and related invention devices may comprise means for activating a sensor on the power and control module 3 or related invention devices. The sensor may take the form of a simple switch, or it may take the form of a more complex sensor. For example, the sensor may be a detector that interacts with the means for activating in such a way that the sensor is capable of detecting a unique identifier composing a part of the endoscopic device that identifies the origin, manufacturer, and/or type of the endoscopic device. Such an identifier, for example, may send a signal to the power and control module. In another embodiment of the invention, the identifier may include a Radio Frequency Identification (RFID) tag or some other integrated-circuit-based identifier mounted anywhere on or otherwise associated with the endoscopic device. In another embodiment of the invention, the identifier may include a resistor mounted on the endoscopic device. In some of these embodiments, the sensor-identifier interaction causes hardware or software in the power and control module to refuse to power the endoscopic device, such as when the power and control module determines that an operator is attempting to inappropriately reuse an endoscopic device.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true, intended, explained, disclose, and understood scope and spirit of this invention's multitudinous embodiments and alternative descriptions.

Additionally, various embodiments have been described above. For convenience's sake, combinations of aspects composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists that specifically excludes that aspect. All patents, test procedures, and other documents cited in this specification are fully incorporated by reference to the extent that this material is consistent with this specification and for all jurisdictions in which such incorporation is permitted.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically exclude the value or the conditions for the aspect.

What is claimed is:

1. An endoscope system comprising:
a reusable receiver comprising circuitry to wirelessly receive electrical image data and output that data to a visualization system
a disposable handpiece remote from the visualization system comprising
a handle;
a conduit extending from near the distal end of the handpiece to near the proximal end of the handpiece;
an imaging system with a mounting portion mounted in the conduit;
a transmitter adapted to transmit wirelessly over regulated or unregulated frequencies electrical image or sensor data to the receiver;
a video camera comprising an image sensor wherein the video camera is in signal communication with the transmitter and wherein the video camera mounts near the proximal end of the imaging system;
and
a power and control module comprising a battery wherein the power and control module is in signal and power connectivity with the disposable handpiece;
wherein the imaging system is adapted to receive an image of a target located near the distal end of the imaging system and to convey that image to the image sensor and wherein the image sensor within the video camera is adapted to convert the image into electrical image data.

2. The endoscope of claim 1 wherein the disposable handpiece further comprises a lumen with an illumination system comprising an LED light source mounted near the distal end of the imaging system or mounted proximal to a light pipe wherein
the light pipe terminates near the distal end of the imaging system and
the distal end of the light pipe is cut and polished at an angle of from about 90° to about 30°.

3. The endoscope of claim 2 wherein frequencies are selected from the Industrial, Scientific and Medical frequency band; the 2.4 GHz frequency band; the 5.8 GHz frequency band; the 900 MHz frequency band; the 2.36 GHz to 2.39 GHz band; or the Wireless Body Area Network frequency band.

4. The endoscope of claim 3 wherein the imaging system comprises a coherent fiber bundle or a gradient-index lens.

5. The endoscope of claim 4 wherein the imaging system comprises two, segmented, coherent fiber bundles, six achromatic optic elements and three dual-lens housings.

6. The endoscope of claim 5 wherein the proximal-most dual-lens housing functions to focus the image.

7. The endoscope of claim 6 wherein at least one coherent fiber bundle has a length of five to nine inches.

8. The endoscope of claim 7 wherein the transmitter is integrated with the video camera.

9. The endoscope of claim 8 wherein the image is transferred to the camera through at least one segmented coherent fiber bundle.

10. The endoscope of claim 8 wherein the power and control module is capable of adjusting the intensity of the light from the illumination system.

11. The endoscope of claim 10 wherein the imaging system is in-line or dual-folded.

12. The endoscope of claim 11 wherein the disposable handpiece further comprises an opening capable of accommodating a surgical tool.

13. The endoscope of claim 11 wherein the power and control module is powered by a disposable or rechargeable battery.

14. The endoscope of claim 13 wherein the power and control module powers the illumination system and the camera.

15. The endoscope of claim 14 wherein sensor data is any one or any combination of carbon dioxide levels, temperature, or fluorescence signals.

16. The endoscope of claim 15 further comprising a lens washing system mounted near the distal end of the imaging system.

17. A method comprising supplying to a surgical facility an endoscope system comprising:
a reusable receiver comprising circuitry to wirelessly receive electrical image data and output that data to a visualization system
a disposable handpiece remote from the visualization system comprising
a handle;
a conduit extending from near the distal end of the handpiece to near the proximal end of the handpiece;
an imaging system with a mounting portion mounted in the conduit;
a transmitter adapted to transmit wirelessly over regulated or unregulated frequencies electrical image or sensor data to the receiver;
a video camera comprising an image sensor wherein
the video camera is in signal communication with the transmitter and wherein
the video camera mounts near the proximal end of the imaging system
and
a power and control module comprising a battery wherein the power and control module is in signal and power connectivity with the disposable handpiece.

18. The method of claim 17 further comprising a step of discarding the handpiece within zero to forty-eight hours after the supplying step.

19. The method of claim 18 further comprising a step of discarding the power and control module within zero to forty-eight hours after the supplying step.

20. An endoscope system comprising:
a reusable receiver comprising circuitry to wirelessly receive electrical image data and output that data to a visualization system
a disposable handpiece remote from the visualization system comprising
a handle;
a conduit extending from near the distal end of the handpiece to near the proximal end of the handpiece;
an in-line or dual-folded imaging system with a mounting portion mounted in the conduit wherein
the imaging system comprises a gradient-index lens or comprises two, segmented, coherent fiber bundles, six achromatic optic elements, and three dual-lens housings;
the proximal-most dual-lens housing functions to focus the image;
at least one coherent fiber bundle has a length of five to nine inches, and
the image is transferred to the camera through at least one segmented coherent fiber bundle
a lumen with an illumination system comprising an LED light source mounted near the distal end of the imaging system or mounted proximal to a light pipe wherein
the light pipe terminates near the distal end of the imaging system and
the distal end of the light pipe is cut and polished at an angle of from about 90° to about 30°
a video camera comprising an image sensor wherein
the video camera is in signal communication with the transmitter and
the video camera mounts near the proximal end of the imaging system
a transmitter adapted to transmit wirelessly over regulated or unregulated frequencies, electrical image or sensor data to the receiver signals wherein
frequencies are selected from the Industrial, Scientific and Medical frequency band; the 2.4 GHz frequency band; the 5.8 GHz frequency band; the 900 MHz frequency band; or the Wireless Body Area Network frequency band, and
wherein sensor data is any one or any combination of carbon dioxide levels, temperatures, or fluorescence signals
an opening in the handle capable of accommodating a surgical tool
a lens washing system mounted near the distal end of the imaging system
and
a disposable- or rechargeable-battery-powered power and control module wherein the power and control module is in signal and power connectivity with the disposable handpiece and powers the camera and the illumination system and is capable of adjusting the intensity of the light from the illumination system
wherein the imaging system is adapted to receive an image of a target located near the distal end of the imaging system and to convey that image to the image sensor and wherein the image sensor within the video camera is adapted to convert the image into electrical image data.
